(19) United States
(12) Patent Application Publication
(10) Pub. No.: US 2004/0181159 A1
(43) Pub. Date: Sep. 16, 2004

(54) Method and apparatus for detecting yin-yang and asthenia-sthenia

(76) Inventors: Terry B.J. Kuo, Ji-An Township (TW); Cheryl C. Yang, Ji-An Township (TW)

Correspondence Address: Harold V. Stotland, Seyfarth Shaw, 42nd Floor, 55 East Monroe Street, Chicago, IL, 60603-5803, US

(21) Appl. No.: 10/770,934

(22) Filed: Feb. 2, 2004

(30) Foreign Application Priority Data
Mar. 11, 2003 (CN) 092105305

(51) Int. Cl. A61B 5/04

(52) U.S. Cl. 600/509

(57) ABSTRACT

A method and an apparatus for detecting yin-yang and asthenia-sthenia by means of the heart rate variability (HRV) analysis, which is originally difficult to be determined and utilized, are revealed, so as to help the clinical diagnoses of the Western and the Chinese physicians. The detecting method comprises the steps of (1) capturing an electrocardiogram signal from a person; (2) converting the electrocardiogram signal into a heart rate power spectral density; (3) computing a low frequency power and a high frequency power of the heart rate power spectral density; (4) calculating a yin parameter and a yang parameter based on the low frequency power and high frequency power; and (5) calculating the standard scores of the yin parameter and the yang parameter to be a yin index and a yang index respectively for determining the yin-yang and asthenia-sthenia for the person.

### BACKGROUND OF THE PRESENT INVENTION

(A) Field of the Present Invention

The present invention is related to a method and an apparatus for detecting yin-yang and asthenia-sthenia, more specifically, to a method and an apparatus for detecting yin-yang and asthenia-sthenia by the heart rate variability (HRV).

(B) Description of the Related Art

The Western medicine believes the autonomic nervous system is in control of the physiological functions of a whole body that are relative to vital maintenance, such as blood pressure, heart rate, windpipe resistance, sweating, body temperature and energy metabolism, and these nervous operations can progress unconsciously. The autonomic nerve falls into the sympathetic and the parasympathetic nerves. In general, the former is relative to circumstance confronting, and the latter is relative to vital maintenance and propagation. For example, if the former is excited, the blood pressure will rise and pupils will magnify, and if the latter is excited, the intestine and stomach will excrete and the penis will erect. In general, both the sympathetic and parasympathetic nerves are vigorous at young age, and deficient at old age. For a male, the sympathetic nerve tends to be vigorous, while the parasympathetic nerve tends to be deficient. On the contrary, for a female, the sympathetic nerve tends to be deficient, while the parasympathetic nerve tends to be vigorous. If the autonomic nervous system is imbalance, it will probably cause various acute and chronic diseases, such as heart disease, hypertension or even sudden death. Therefore, the health care of the autonomic nerve is an important subject for medical sciences.

Recently, quite a number of techniques for diagnosing the functions of the autonomic nerve are successfully developed in succession. These days, by the advancement of the computer science and the spectral analysis technique, the functions of the autonomic nerve can be detected and quantified by the subtle fluctuation of heart rate, namely heart rate variability (HRV), while a person rests. In other words, the function of the autonomic nerve can be analyzed or diagnosed without interrupting the work and the rest of a person. By employing the spectral analysis, researchers found that the subtle fluctuations of HRV can be explicitly divided into two types, namely a high-frequency (HF) component and a low-frequency (LF) component. The HF component is synchronous to the respiratory rate, and thus it is also called respiratory-related component. The LF component is estimated to be relative to the vasomotion or baroreceptor reflex. Some investigators further divide the LF component into a very-low-frequency (VLF) component and a low-frequency component.

Many physiologists have already found that the HF component of heart rate or the total power of HRV can represent the functions of the vagus nerve (the parasympathetic nerve) of a heart, and the ratio of the LF component to the HF component can show the activity of the sympathetic nerve of the heart. Previous researches have found that the HRV also can represent many physiological functions. For instance, if the intra cranial pressure of a patient rises, the total power of the HRV will decrease. According to the public health investigation of USA Framingham, if the LF component of an old person's heart rate decreases by one standard deviation, the mortality for that old person is 1.7 times that of a normal one.

To date, a series of software and hardware for real-time and on-line spectral analysis of physiological signals have been developed. For instance, the LF component of heart rate or blood pressure is as the index of the extent of anaesthetization. In an intensive care unit, it can be found that the livability decreases with the lower HRV of a patient, the LF component of the HRV of a brain-dead patient is gone, and if the repulsion phenomenon happens to a heart exchange patient, the HRV will change accordingly.

Yin-yang is an important theoretical basis and thinking logic in traditional Chinese medicine, and is used for explaining the symptoms of diseases. For instance, yin-asthenia may cause dry throat and mouth, night sweat, etc., yang-asthenia may result in hypodynamia, breath difficulty, etc., and many diseases may result from the imbalance of the yin-yang. The combinations of the yin, yang, asthenia and sthenia can be categorized into four symptoms, yin-sthenia and yang-sthenia, yin-sthenia and yang-asthenia, yin-asthenia and yang-sthenia, and both yin and yang being in asthenia. Accordingly, corresponding cure methods have been applied to different symptoms, recognized by the masses for thousands of years and have been helping numerous of patients relieve from the diseases.

Though the theories of Chinese medicine and Western medicine are tremendously different from each other, the Western medicine believes the autonomic nerve is in control of the physiological functions of a body that are relative to vital maintenance, in which sympathetic nerve and parasympathetic nerve are also either in confronting or in cooperation that is common with the yin-yang movement of the Chinese medicine. By applying the yin-yang and qi-asthenia, qi-sthenia theory of Chinese medicine, which is most similar to the sympathetic nerve and the parasympathetic nerve, in clinic, it helps analyze the parameters of autonomic nerve so that the health care of autonomic nerve system can be promoted easily. However, the diagnosis for yin-yang, qi-asthenia and qi-sthenia so far still relies on the judgment of an experimented Chinese physician, as few equipments can assist the physician for diagnosis, i.e., the quantified data is insufficient for the objective evaluation by the physician.

### SUMMARY OF THE INVENTIION

The objective of the present invention is to provide a method and an apparatus for detecting yin-yang and asthenia-sthenia by means of the HRV analysis, which is originally difficult to determine and utilize, so as to help the diagnoses by Western and Chinese physicians.

The detecting method for the yin-yang, qi-asthenia and qi-sthenia of the present invention includes the steps of (1) capturing an electrocardiogram (ECG) signal of a person; (2) converting the electrocardiogram signal into a heart rate power density spectral (HPSD) by Fourier transformation; (3) calculating the LF power and HF power of the HRV power density spectral; (4) obtaining a yin parameter and a yang parameter by the LF power and HF power; and (5) calculating a yin index, a yang index and a qi index of the person, wherein the yin index and yang index are the standard score of the yin parameter and yang parameter, respectively, and the qi index can be obtained by adding the yin index to the yang index or be the standard score of the LF power for determining the yin-yang and asthenia-sthenia.

The above-mentioned yin parameter is the HF power or the summation of the HF and the LF powers, and the yang parameter is the ratio of the LF power to the HF power or the percentage of LF power of the summation of the HF and LF powers.

For a person, if the number that the yang index minus the yin index equals to zero, the yin and the yang are completely balanced. If the number is positive, it represents that the person is in yang status, and the larger the number is, and the more yang there is. If the number is negative, it represents that the person is in the yin status, and the smaller the number is, the more yin there is. In addition, if the qi index is zero, it represents that the asthenia and sthenia are in equilibrium. If it is positive, it represents that the person is in the qi-sthenia status, and the large the number is, the more qi-sthenia there is. If it is negative, it represents the person is in the qi-asthenia status, and the smaller the number is, the more qi-asthenia there is. Therefore, the quantified yin-yang and asthenia-sthenia indexes can be obtained.

The apparatus for detecting the yin-yang and asthenia-sthenia can be implemented by a heartbeat sensor and a computer, the heartbeat sensor being used for capturing the ECG signal, the computer containing an analog-to-digit converter (ADC) and a program, wherein the ADC can digitize the ECG signal, and the program can convert the ECG signal to the power spectrogram and calculate the LF power and the HF power so as to further obtain the yin index, yang index and qi index of the ECG signal.

The heartbeat sensor can employ electrodes, a pressure sensor, a microphone or a photodiode, etc.

### BRIEF DESCRIPTION OF THE DRAWINGS

FIG. 1 illustrates the apparatus for detecting yin-yang and asthenia-sthenia of the present invention;

FIG. 2 illustrates the QRS wave of the ECG signal used in sampling of the present invention;

FIG. 3 illustrates the method for detecting yin-yang and asthenia-sthenia of the present invention;

FIG. 4, FIG. 6, FIG. 7 and FIG. 8 illustrate the two-dimensional diagrams and Taichi figures of various individuals employing the present invention; and

FIG. 5 is the flow chart of the method for detecting yin-yang and asthenia-sthenia of the present invention.

### DETAILED DESCRIPTION OF THE PRESENT INVENTION

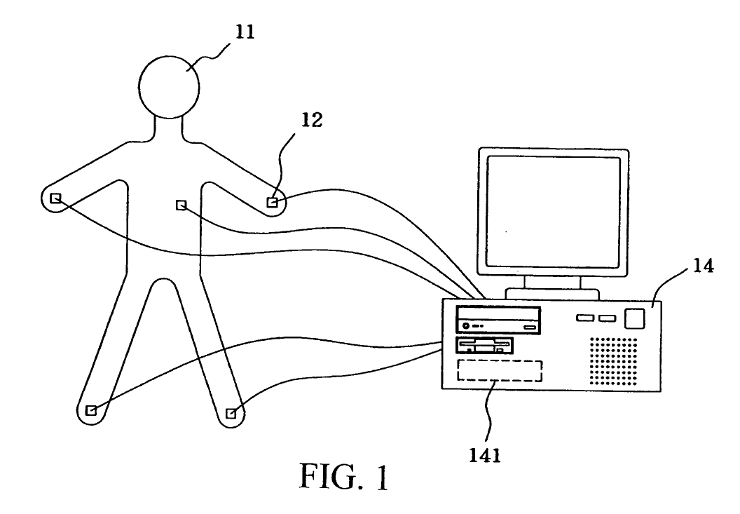

Referring to FIG. 1, the embodiment of the present invention uses electrodes 12 as a heartbeat sensor to collect an ECG signal of a human body 11. The ECG signal are input to a computer 14 after being amplified 1000 times and filtered by band-pass of 0.16-16 Hz, and then an ADC 141 contained in the computer 14 samples the ECG signal by the rate of 256 times per second. The digitized ECG signal can be immediately analyzed by an on-line program of the computer 14 to acquire the yin-yang and asthenia-sthenia data of the human body 11, and the result can be stored in the computer 14 for off-line analysis or as a record. The electrodes 12 can also be replaced with a pressure sensor, a microphone or a photodiode as long as they have the capability of detecting the ECG signal. This embodiment only uses the computer 14 including the ADC 141 to store and analyze the ECG signal. Therefore, valuable auxiliary data can be obtained at very low cost.

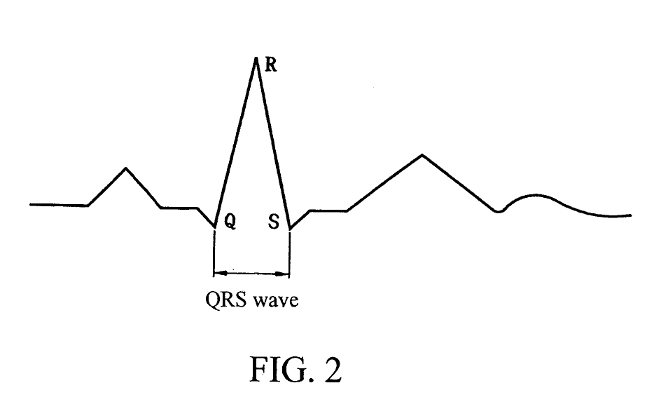

The ECG signal has to be filtered to remove noises before being sampled; the filtering process is described as follows. FIG. 2 illustrates an ECG signal of a heartbeat, the most bulgy wave segment being called a QRS wave, wherein the first upper deflection point is designated as Q, the peak as R, and the final bottom end as S. In the process of QRS identification, first of all, the peak detection program is employed to find out the QRS wave in the ECG signal, and the parameters of the amplitude and the duration of the QRS wave are measured. Secondly, the mean and standard deviation of the parameters are calculated as a standard template, and each subsequent QRS wave is compared to the standard template. If the comparison result shows that a QRS wave falls out three standard deviation of the standard template, it will be regarded as a noise or an ectopic beat so as to be deleted. The R point of a qualified QRS wave is selected as the timing of a heartbeat, and the period between the heartbeat and the next one is as the period of the heartbeat (R-R interval). Afterward, the filter procedure for R-R interval is executed, in which the mean and standard deviation of all R-R intervals are calculated, and then all R-R intervals are filtered. If an R-R interval is out of the range for four standard deviations, it will be regarded as an error or an instable signal and be deleted.

Sequentially, the qualified ECG signal is sampled by the rate of 7.11 Hz and interpolated to keep the time consecution. First, the linear drift of the ECG is eliminated to prevent the disturbance of the LF band, and Hamming computing is employed to avoid the mutual leakages of individual frequency components. Secondly, 288 seconds data (2048 points) of the ECG signal were performed “fast Fourier transform” to obtain the heart rate power spectral density (HPSD), and then the affections caused by sampling and Hamming computing is compensated to reduce deviation.

The powers of the two frequency bands are quantified by the integral of the HPSD, including the LF power between 0.04 to 0.15 Hz and the HF power between 0.15 to 0.4 Hz. Moreover, the total power (TP) of the HF and the LF powers, the ratio of the LF power to the HF power (LF/HF) and the percentage of LF in TP (LF %) are computed as well, and the HF power or TP relative to the activity of the heart parasympathetic nerve is defined as a yin parameter, and the LF/HF or LF % relative to the activity of the heart sympathetic nerve is defined as a yang parameter, and the LF power is regarded as the combinational index of the sympathetic and parasympathetic nerve functions, i.e., the main index of the autonomic nerve function, which is relative to the asthenia-sthenia in Chinese medicine.

Before evaluating the yin-yang and asthenia-sthenia, the following definitions are determined as:

1. Standard score SS(χ)=(χ−mean χ)/SD χ, wherein mean χ, SD χ respectively represents the mean and the standard deviation of χ, the same definition with the statistics.

2. Yin and yang indexes:

Yin index=SS(HF) or SS(TP); and

Yang index=SS(LF/HF) or SS(LF %).

For a person, if the value that the yang index minus the yin index equals to zero, then yin and yang are completely balanced. If the value is positive, it represents the person is in yang status, and the larger the value is, the more yang there is. On the contrary, if the value is negative, it represents the person is in yin status, and the smaller the value is, the more yin there is.

In addition, the yang index plus yin index or SS (LF) can be a qi index. If the qi index is zero, it represents the asthenia-sthenia is normal. If it is positive, it represents the individual is in qi-sthenia status, and the larger the value is, and the more qi-sthenia there is. If it is negative, it represents the individual is in qi-asthenia status, and the smaller the value is, the more qi-asthenia there is.

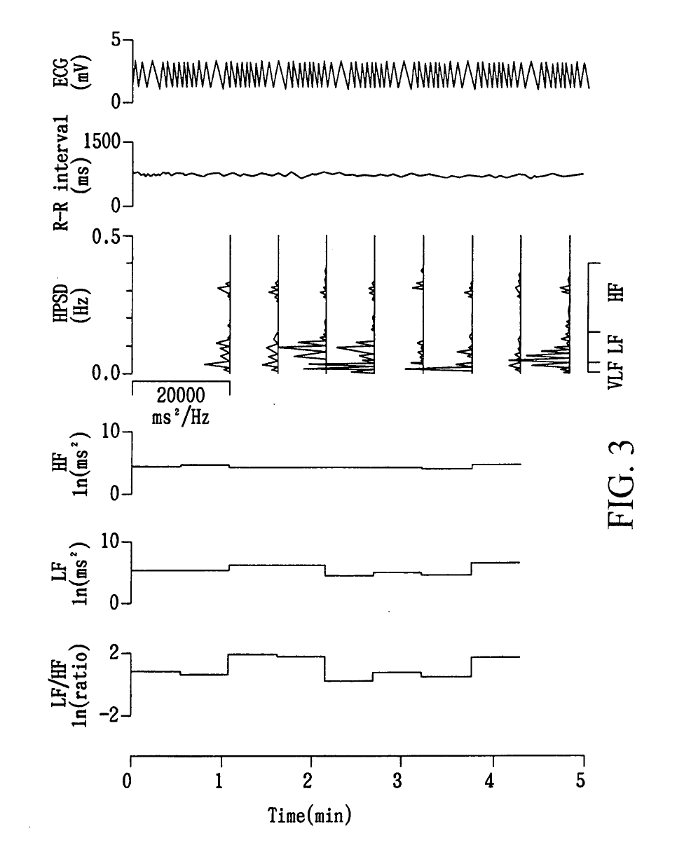

Referring to FIG. 3, a cardiogram testing is performed on a forty-year-old male for five minutes, and the ECG signal and R-R interval are recorded. The result is shown in the right portion of FIG. 3. After the ECG signal passes the QRS and R-R interval filtering, Flourier transformation is performed on each time period so as to obtain the HPSD. Based on frequency, the HPSD is divided into the VLF band of between 0.003 and 0.04 Hz, the LF band of between 0.04 and 0.15 Hz and the HF band of between 0.15 and 0.4 Hz. Afterward, the corresponding power of the HPSD at different frequency band and time are summed up, i.e., integrate, to respectively acquire the HF power, the LF power and the LF to HF power ratio (LF/HF). For the sake of the brevity and the correctness of figures, the values of the parameters in FIG. 3 are in logarithm scale. The means of the HF, the LF/HF and the LF in logarithm scale are 4.41, 1.06 and 5.47 respectively, and the logarithm of the LF power percentage of TP is 62.5.

Before computing the standard score of the above parameters, a database has to be built up based on the same procedure beforehand, and it records values such the HF, LF/HF, LF % parameters of healthy people, patients, and people of various ages and genders to obtain the mean and the standard score. The mean of each parameter is as the mean χ of the standard score (SS function). The standard deviation of each parameter is as the SD χ of standard score. The standard score of each parameter is counted according to different ages, genders and symptoms, and is categorized into the yin-yang and asthenia-sthenia for the following comparison.

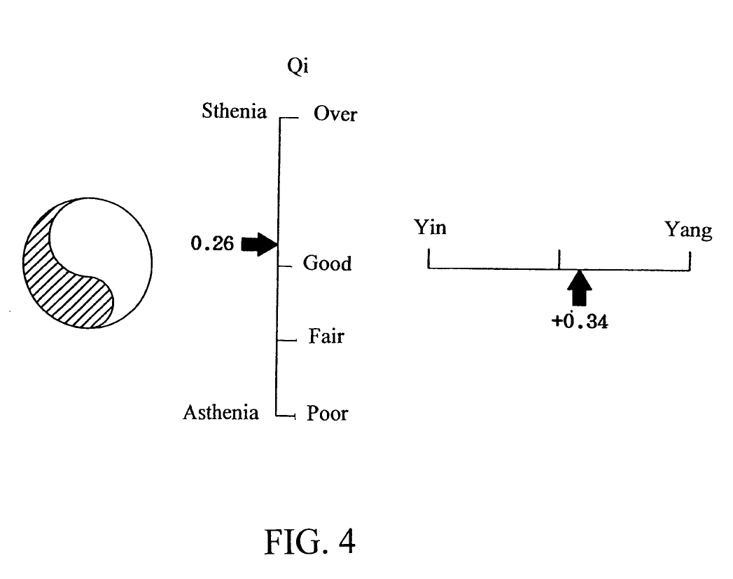

In this embodiment, the yin index, the yang index and the qi index are respectively obtained by substituting corresponding variables such as the HF, LF % and LF into the SS function, and are 0.0322 [symbol] 0.3689 and 0.2588 respectively. The yang index minus yin index equals 0.3366. The SS(LF) and the number that the yang index minus the yin index are inserted into the yin-yang and asthenia-sthenia database to build a two-dimensional diagram shown as FIG. 4, in which the abscissa represents the yin-yang and the ordinate represents the qi (the asthenia and the sthenia represent the vigorous qi and the deficient qi respectively). If the exponential SS(LF) or exponential (yang index plus yin index) is as the diameter of a circle, and the ratio of the exponential yin index and the exponential yang index are as the ratio of black (shadow) to white in the circle, a Taichi figure can be made as shown in the left portion of FIG. 4. The diameter of the Taichi figure represents qi and they are in directly proportional relation, and the black and the white portions represent the yin and the yang respectively. Based on the two-dimensional diagram or the Taichi figure in FIG. 4, the qi is in adequacy and is partial to the yang status, i.e., the functions of autonomic nerve are adequate, and the person leans to the parasympathetic nerve.

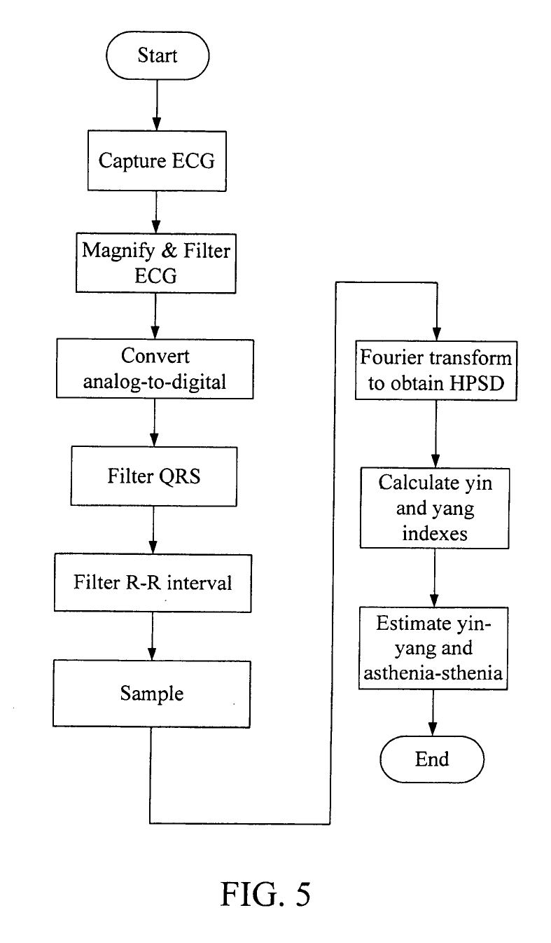

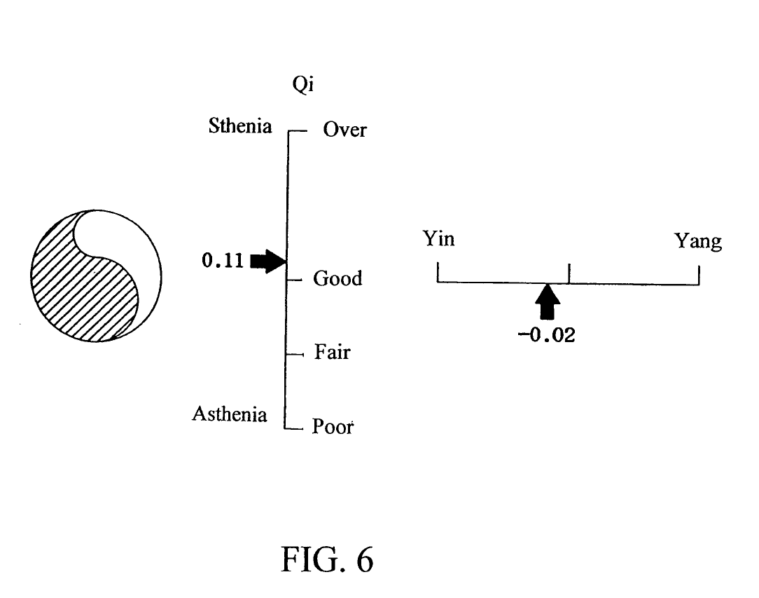

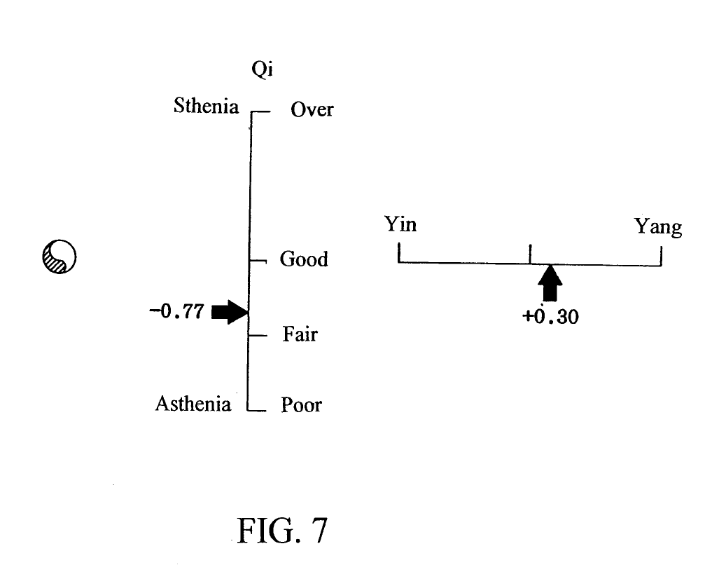

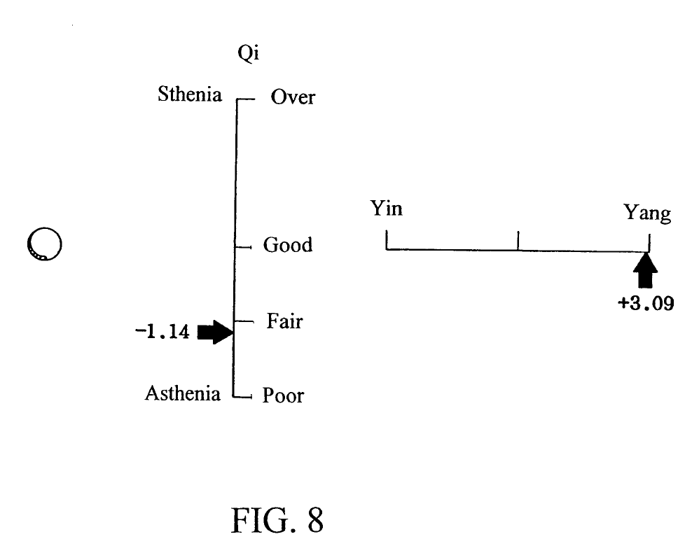

The procedure of detecting the yin-yang and asthenia-sthenia is shown in FIG. 5. First, a heartbeat sensor is used for capturing an ECG signal, and the ECG signal is digitized, followed by magnification and filtering. Secondly, the ECG signal is filtered based on the standard QRS wave and R-R interval, the ECG signal that exceeds three or four standard deviations are deleted. Sequentially, the ECG signal is sampled and performed Flourier transformation to obtain the HPSD, then the HF and LF powers are computed so as to further obtain the yin-yang and qi indexes for the yin-yang and asthenia-sthenia evaluation.

Besides, ECG analysis is also performed on a forty-year-old female, a seventy-eight-year-old male, a fifty-nine-year-old female with diabetes mellitus and a brain-dead, fifty-three-year-old male patient according to the above procedure, the outcomes are summarized in the following table:

| | | | | 59 years | 53 years |
|---|---|---|---|---|---|
| | 40 years | 40 years | 78 years | old | old |
| | old | old | old | (female | (brain- |
| | (healthy | (healthy | (healthy | with | dead |
| | male) | female) | male) | diabetic) | male) |
| LF | 5.47 | 5.47 | 3.23 | 3.34 | 0.494 |
| HF | 4.41 | 5.04 | 3.08 | 1.32 | 4.03 |
| LF % | 62.5 | 47.3 | 37.6 | 55.8 | 2.27 |
| LF/HF | 1.06 | 0.428 | 0.149 | 2.01 | −3.5 |
| Yang | 0.3689 | −0.171 | −0.148 | 0.6051 | −3.31 |
| SS(LF %) | | | | | |
| Yin | 0.0322 | 0.1483 | −0.452 | −2.48 | −0.071 |
| SS(HF) | | | | | |
| Yin-Yang | 0.3366 | −0.3193 | 0.304 | 3.0851 | −3.239 |
| Balance | | | | | |
| SS(LF %)- | | | | | |
| SS(HF) | | | | | |
| Qi | 0.2588 | 0.1079 | −0.767 | −1.14 | −4.99 |
| SS(LF) | | | | | |

The two-dimensional diagram and Taichi figure of the forty years old female are shown in FIG. 6, she is interpreted as in adequate qi-sthesia, and is partial to the yin status, i.e., the autonomic nerve functions adequately and is partial to the parasympathetic nerve. The two-dimensional diagram and Taichi figure of the seventy-eight-year-old male is shown in FIG. 7, he is interpreted as in adequate qi-asthenia, and the yin and yang are balanced, i.e., the autonomic nerve functions adequately and belongs to the neutral status. The two-dimensional diagram and Taichi figure of the fifty-nine-year-old female is shown in FIG. 8, she is interpreted as in excessive qi-asthenia and in excessive yang status, i.e., the autonomic nerve functions inadequately and mainly directed by the sympathetic nerve. As to the brain-dead, fifty-three-year-old male patient, because his LF value approaches zero, the diameter of the corresponding Taichi figure is very small, and is thus omitted. Moreover, through the comparison of the yin, yang and qi indexes to standard values, he is interpreted as being in pure yin status, and the qi is exhausted, i.e., the autonomic nerve is diagnosed as prostrated in accordance with the west medicine. Obviously, the result of the west medicine nearly complies with that of the Chinese medicine.

From the above descriptions, a person's yin-yang and asthenia-sthenia, corresponding to the functions of the autonomic nerve and the sympathetic and parasympathetic nerves in Western medicine, can be determined by the two-dimensional diagram or the Taichi figure, and they can act as objective data to assist clinical diagnosis. In the embodiment of the present invention, it is found that functions of the sympathetic and the parasympathetic nerves are similar to the yin-yang and qi-asthenia, qi-sthenia of the Chinese medicine.

The above-described embodiments of the present invention are intended to be illustrative only. Numerous alternative embodiments may be devised by those skilled in the art without departing from the scope of the following claims.

What is claimed is:

1. A method for detecting yin-yang and asthenia-sthenia, comprising the steps of:
capturing an electrocardiogram signal from a person;
converting the electrocardiogram signal into a heart rate power spectral density;
computing a low frequency power and a high frequency power of the heart rate power spectral density;
calculating a yin parameter and a yang parameter based on the low frequency power and the high frequency power; and
calculating standard scores of the yin parameter and the yang parameter to act as a yin index and a yang index respectively for determining the yin-yang and asthenia-sthenia of the person.

2. The method for detecting yin-yang and asthenia-sthenia of claim 1, wherein the yin parameter is one of the high frequency power and the summation of the high and low frequency powers.

3. The method for detecting yin-yang and asthenia-sthenia of claim 1, wherein the yang parameter is one of the ratio of the low frequency power to the high frequency power and the percentage of the low frequency power to the summation of the low and high frequency powers.

4. The method for detecting yin-yang and asthenia-sthenia of claim 1, further comprising the step of subtracting the yin index from the yang index for determining the yin-yang balance of the person.

5. The method for detecting yin-yang and asthenia-sthenia of claim 1, further comprising the step of calculating the standard score of the low frequency power for determining the asthenia-sthenia of the person.

6. The method for detecting yin-yang and asthenia-sthenia of claim 1, further comprising the step of adding the yang and yin indexes for determining the asthenia-sthenia of the person.

7. The method for detecting yin-yang and asthenia-sthenia of claim 1, wherein the standard score SS(χ)=(χ−mean χ)/SD χ, the mean χ, SD χ respectively represents the mean and the standard deviation of χ, and the χ is one of the yin parameter and the yang parameter.

8. The method for detecting yin-yang and asthenia-sthenia of claim 1, further comprising the step of making a two-dimensional diagram, wherein the yang index minus yin index is placed in the abscissa of the two-dimensional diagram, and the standard score of the low frequency power is placed in the ordinate of the two-dimensional diagram.

9. The method for detecting yin-yang and asthenia-sthenia of claim 1, further comprising the step of making a Taichi figure, wherein the diameter of the Taichi figure is in direct proportion to the exponential standard score of the low frequency power, and the proportion of the yin and the yang of the Taichi figure is equivalent to the ratio of the exponential yang index to the exponential yin index.

10. The method for detecting yin-yang and asthenia-sthenia of claim 1, further comprising the step of making a Taichi figure, wherein the diameter of the Taichi figure is in direct proportion to the low frequency power, and the proportion of the yin and the yang of the Taichi figure is equivalent to the ratio of the exponential yang index to the exponential yin index.

11. The method for detecting yin-yang and asthenia-sthenia of claim 1, wherein the heart rate power spectral density is obtained by Fourier transforming the heartbeat intervals.

12. The method for detecting yin-yang and asthenia-sthenia of claim 1, further comprising the step of filtering QRS waves and heartbeat intervals.

13. An apparatus for detecting yin-yang and asthenia-sthenia, comprising:
a heartbeat sensor for capturing an electrocardiogram signal; and
a computer, including:
an analog-to-digital converter for digitizing the electrocardiogram signal; and
a program for converting the electrocardiogram signal into a heart rate power spectral density, and calculating the low and high frequency powers to acquire a yin index, a yang index and a qi index of the heartbeat.

14. The apparatus for detecting yin-yang and asthenia-sthenia of claim 13, wherein the program can make a Taichi figure, the diameter of the Taichi figure is in direct proportion to the low frequency power, and the proportion of the yin and the yang of the Taichi figure is equivalent to the ratio of the exponential yang index to the exponential ying index.

15. The apparatus for detecting yin-yang and asthenia-sthenia of claim 13, wherein the program can make a two-dimensional figure, the yang index minus yin index is placed in the abscissa of the two-dimensional diagram, and the standard score of the low frequency power is placed in the ordinate of the two-dimensional diagram.
